(19) United States
(12) Patent Application Publication
(10) Pub. No.: US 2021/0298854 A1
(43) Pub. Date: Sep. 30, 2021

(54) ROBOTICALLY-ASSISTED SURGICAL DEVICE, ROBOTICALLY-ASSISTED SURGICAL METHOD, AND SYSTEM

(71) Applicants: Medicaroid Corporation, Kobe-shi (JP); Ziosoft, Inc., Tokyo (JP)

(72) Inventors: Jota IDA, Kobe-shi (JP); Mitsuichi HIRATSUKA, Kobe-shi (JP); Shusuke CHINO, Tokyo (JP); Tsuyoshi NAGATA, Tokyo (JP); Yutaka KARASAWA, Tokyo (JP); Shinichiro SEO, Tokyo (JP)

(21) Appl. No.: 17/209,891

(22) Filed: Mar. 23, 2021

(30) Foreign Application Priority Data
Mar. 26, 2020 (JP) 2020-055964

(51) Int. Cl. A61B 34/00; A61B 34/30

(52) U.S. Cl. CPC A61B 34/70; A61B 2034/301; A61B 34/30

(57) ABSTRACT

A robotically-assisted surgical device assists endoscopic surgery by a surgical robot. The robotically-assisted surgical device includes a processor. The processor is configured to derive a positional relationship between the surgical robot and an access platform installed on a subject which is a surgery target and capable of inserting at least two surgical instruments.

### CROSS-REFERENCE TO RELATED APPLICATIONS

This application is based on and claims priority under 35 USC 119 from Japanese Patent Application No. 2020-055964 filed on Mar. 26, 2020, the contents of which are incorporated herein by reference.

### TECHNICAL FIELD

The present disclosure relates to a robotically-assisted surgical device, a robotically-assisted surgical method, and a system.

### BACKGROUND ART

In the related art, transanal minimally invasive surgery (TAMIS) is known as one of surgical procedures. In TAMIS, it is known to install a platform (TAP: Transanal Access Platform) in an anus of a patient in order to insert a surgical instrument into the patient (refer to GelPOINT Path, Transanal Access Platform, Applied Medical, searched on Dec. 26, 2019, Internet <URL: https://www.appliedmedical.com/Products/Gelpoint/Path>).

Since the platform used in TAMIS is not attached to the bone or the like of the patient, the platform easily moves with respect to the patient during surgery. For example, a rectum which is a surgery target easily moves following the movement of the platform.

Therefore, for example, an operating range of the surgical instrument in the body of the patient may be limited. In addition, the surgical instrument may receive a pressing force from the moving platform. Therefore, the movement of the platform may reduce the operability and safety of surgery using surgical instruments.

In view of the above-described circumstances, the present disclosure provides a robotically-assisted surgical device, a robotically-assisted surgical method, and a system that can improve the operability and safety in surgery using a platform installed on a subject.

### SUMMARY

A robotically-assisted surgical device related to one aspect of the present disclosure assists endoscopic surgery by a surgical robot. The robotically-assisted surgical device includes a processor. The processor is configured to derive a positional relationship between the surgical robot and an access platform installed on a subject which is a surgery target and capable of inserting at least two surgical instruments.

According to the present disclosure, it is possible to improve the operability and safety in surgery using a platform installed on a subject.

### BRIEF DESCRIPTION OF DRAWINGS

Exemplary embodiment(s) of the present invention will be described in detail based on the following figures, wherein:

FIG. 1A is a block diagram illustrating a configuration example of a robotically-assisted surgical system according to a first embodiment;

FIG. 1B is a block diagram illustrating a hardware configuration example of a robotically-assisted surgical device;

FIG. 2A is a block diagram illustrating a functional configuration example of the robotically-assisted surgical device;

FIG. 2B is a view illustrating a structural example of a platform;

FIG. 3A is a view for describing a first derivation example of a positional relationship between a surgical robot and the platform;

FIG. 3B is a view for describing a second derivation example of the positional relationship between the surgical robot and the platform;

FIG. 3C is a view illustrating that a body position of a subject is a jackknife position;

FIG. 4 is a view illustrating an example of a state of the platform, a surgical instrument, and the inside of the subject;

FIG. 5 is a view illustrating a rotation example of the surgical instrument around the platform;

FIG. 6 is a view illustrating an example of a state where the surgical instrument inserted through the platform is curved;

FIG. 7 is a view illustrating a movement example of an organ which is in conjunction with movement of the platform;

FIG. 8 is a flowchart illustrating an operation example of the robotically-assisted surgical device;

FIG. 9 is a flowchart illustrating an operation example of the robotically-assisted surgical device (continued from FIG. 8); and

FIG. 10 is a flowchart illustrating an example of an installation simulation procedure of the platform.

### DESCRIPTION OF EMBODIMENTS

Hereinafter, embodiments of the present disclosure will be described with reference to the drawings.

### First Embodiment

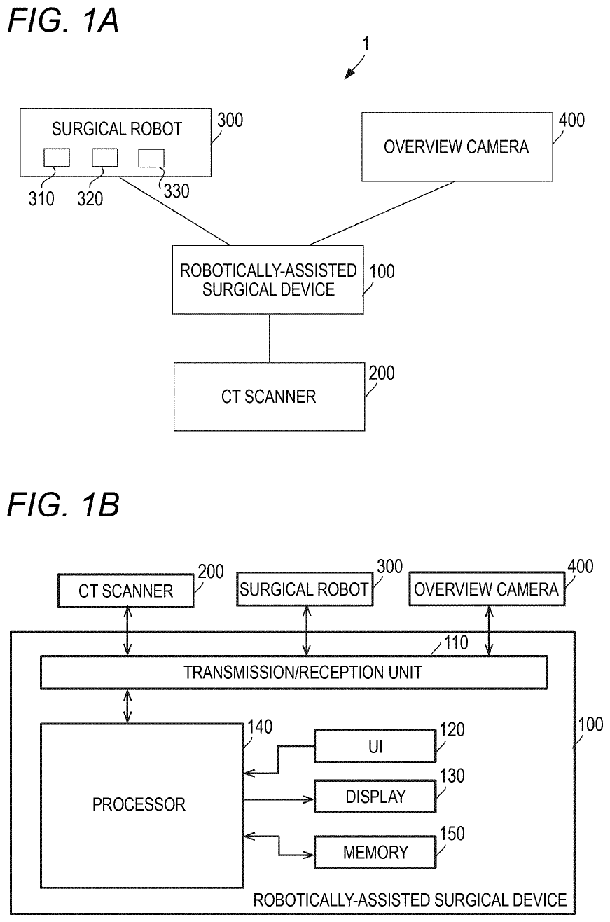

FIG. 1A is a block diagram illustrating a configuration example of a robotically-assisted surgical system 1 according to a first embodiment. The robotically-assisted surgical system 1 includes a robotically-assisted surgical device 100, a CT scanner 200, a surgical robot 300, and an overview camera 400. The robotically-assisted surgical device 100, the CT scanner 200, the surgical robot 300, and the overview camera 400 may be connected to each other via a network. The robotically-assisted surgical device 100 may be connected to each device of the CT scanner 200, the surgical robot 300, and the overview camera 400 on a one-to-one basis. FIG. 1A exemplifies that the robotically-assisted surgical device 100 is connected to each of the CT scanner 200, the surgical robot 300, and the overview camera 400.

The robotically-assisted surgical device 100 acquires various pieces of data from the CT scanner 200, the surgical robot 300, and the overview camera 400. The robotically-assisted surgical device 100 performs image processing based on the acquired data to assist the robotic surgery by the surgical robot 300. The robotically-assisted surgical device 100 may be configured of a PC and software installed in the PC. The robotically-assisted surgical device 100 performs surgery navigation. The surgery navigation includes, for example, preoperative simulation for performing planning before surgery (preoperative planning) and intraoperative navigation for performing the assistance during surgery.

The CT scanner 200 irradiates the subject with X-rays, and captures images (CT images) by using the difference in X-ray absorption by tissues in the body. The subject may include a living body, a human body, an animal, and the like. The CT scanner 200 generates the volume data including information on any location on the inside of the subject. The CT scanner 200 transmits the volume data as the CT image to the robotically-assisted surgical device 100 via a wired circuit or a wireless circuit. Imaging conditions for CT images or contrast conditions for administration of a contrast medium may be taken into consideration when capturing CT images.

The surgical robot 300 includes a robot operation terminal 310, a robot main body 320, and an image display terminal 330.

The robot operation terminal 310 includes a hand controller and a foot switch operated by an operator. The robot operation terminal 310 operates a plurality of robot arms AR provided in the robot main body 320 in response to the operation of the hand controller or the foot switch by the operator. The robot operation terminal 310 includes a viewer. The viewer may be a stereo viewer, and may display a three-dimensional image by fusing the images captured by an endoscope ES (endoscope camera). The plurality of robot operation terminals 310 may exist, and the robotic surgery may be performed by a plurality of operators operating the plurality of robot operation terminals 310.

The robot main body 320 includes the plurality of robot arms AR for performing the robotic surgery, an end effector EF (forceps, instruments) attached to the robot arm AR, and the endoscope ES attached to the robot arm AR. Since the end effector EF and the endoscope ES are used for endoscopic surgery, the end effector EF and the endoscope ES are also referred to as surgical instruments 30 in the embodiment. The surgical instrument 30 includes at least one of one or more end effectors EF and endoscopes ES.

The robot main body 320 is provided with, for example, four robot arms AR, and includes a camera arm to which the endoscope ES is attached, a first end effector arm to which the end effector EF operated by the hand controller for the right hand of the robot operation terminal 310 is attached, a second end effector arm to which the end effector EF operated by the hand controller for the left hand of the robot operation terminal 310 is attached, and a third end effector arm to which the end effector EF for the replacement is attached. Each robot arm AR has a plurality of joints, and may be provided with a motor and an encoder corresponding to each joint. The encoder may include a rotary encoder as an example of an angle detector. Each robot arm AR has at least 6 degrees of freedom, preferably 7 or 8 degrees of freedom, and may operate in the three-dimensional space and be movable in each direction within the three-dimensional space. The end effector EF is an instrument that actually comes into contact with the treatment target in a subject PS in the robotic surgery, and enables various treatments (for example, grasping, excision, peeling, and suturing).

The end effector EF may include, for example, grasping forceps, peeling forceps, an electric knife, and the like. As the end effector EF, a plurality of separate end effector EFs different for each role may be prepared. For example, in the robotic surgery, the tissue may be suppressed or pulled by two end effector EFs, and the tissue may be cut by one end effector EF. The robot arm AR and the surgical instrument 30 may operate based on an instruction from the robot operation terminal 310. At least two end effectors EF are used in the robotic surgery.

The image display terminal 330 has a monitor and a controller for processing the image captured by the endoscope ES, and displaying the image on a viewer or a monitor. The monitor is confirmed by, for example, a robotic surgery assistant or a nurse.

The surgical robot 300 performs the robotic surgery in which an operation of the hand controller or the foot switch of the robot operation terminal 310 by the operator is received, the operations of the robot arm AR, the end effector EF, and the endoscope ES of the robot main body 320 are controlled, and various treatments for the subject PS are performed. In the robotic surgery, the endoscopic surgery may be performed in the subject PS.

In the embodiment, it is mainly assumed that the transanal minimally invasive surgery (TAMIS) is performed using the surgical robot 300. In TAMIS, a platform 40 (TAP) is installed on the anus of the subject PS in order to insert the surgical instrument 30 into the subject PS. In TAMIS, since the platform 40 is installed on the anus, which is a hole of the subject PS, it is not necessary to perforate a port on the body surface of the subject PS unlike installation of a trocar. In TAMIS, gas may be injected through the platform 40 to inflate the tissues or organs existing in the neighborhood of the anus of the subject PS. The tissues or organs existing in the neighborhood of the anus of the subject PS may include, for example, rectum, colon, prostate, and the like. The platform 40 has a valve and maintains the inside of the subject PS airtight. Air (for example, carbon dioxide) may be continuously introduced into the subject PS for maintaining the airtight state.

The end effector EF is inserted through the platform 40. The valve of the platform 40 is opened when the end effector EF is inserted, and the valve of the platform 40 is closed when the end effector EF is detached. The end effector EF is inserted via the platform 40, and various treatments are performed depending on the surgical procedure. The robotic surgery may be applied to the endoscopic surgery of other parts (for example, palatal jaw surgery or mediastinal surgery) in addition to a case where the organs neighbor of the anus are surgery targets.

FIG. 1B is a block diagram illustrating a configuration example of the robotically-assisted surgical device 100. The robotically-assisted surgical device 100 includes a transmission/reception unit 110, a UI 120, a display 130, a processor 140, and a memory 150.

The transmission/reception unit 110 includes a communication port, an external device connection port, a connection port to an embedded device, and the like. The transmission/reception unit 110 acquires various pieces of data from the CT scanner 200, the surgical robot 300, and the overview camera 400. The various pieces of acquired data may be immediately sent to the processor 140 (processing unit 160) for various types of processing, or may be sent to the processor 140 for various types of processing when necessary after being stored in the memory 150. The various pieces of data may be acquired via a recording medium or a storage medium.

The transmission/reception unit 110 transmits various pieces of data to the CT scanner 200, the surgical robot 300, and the overview camera 400. The various pieces of data to be transmitted may be directly transmitted from the processor 140 (processing unit 160), or may be transmitted to each device when necessary after being stored in the memory 150. The various pieces of data may be sent via a recording medium or a storage medium.

The transmission/reception unit 110 may acquire volume data from the CT scanner 200. The volume data may be acquired in the form of intermediate data, compressed data or sinogram. The volume data may be acquired from information from a sensor device attached to the robotically-assisted surgical device 100.

The transmission/reception unit 110 acquires information from the surgical robot 300. The information from the surgical robot 300 may include information on the kinematics of the surgical robot 300. The information on the kinematics may include, for example, shape information regarding the shape and motion information regarding motion of an instrument (for example, the robot arm AR, the end effector EF, the endoscope ES) for performing the robotic surgery included in the surgical robot 300. The information on the kinematics may be received from an external server.

The shape information may include at least a part of information such as the length and weight of each part of the robot arm AR, the end effector EF, and the endoscope ES, the angle of the robot arm AR with respect to the reference direction (for example, a horizontal surface), and the attachment angle of the end effector EF with respect to the robot aim AR.

The motion information may include the movable range in the three-dimensional space of the robot arm AR, the end effector EF, and the endoscope ES. The motion information may include information such as the position, speed, acceleration, or orientation of the robot arm AR when the robot arm AR operates. The motion information may include information such as the position, speed, acceleration, or orientation of the end effector EF with respect to the robot arm AR when the end effector EF operates. The motion information may include information such as the position, speed, acceleration, or orientation of the endoscope ES with respect to the robot arm AR when the endoscope ES operates.

An angle sensor may be attached to the robot arm AR, the end effector EF, or the endoscope ES. The angle sensor may include a rotary encoder that detects an angle corresponding to the orientation of the robot arm AR, the end effector EF, or the endoscope ES in a three-dimensional space. The transmission/reception unit 110 may acquire the detection information detected by various sensors attached to the surgical robot 300.

The transmission/reception unit 110 may acquire operation information regarding the operation with respect to the robot operation terminal 310. The operation information may include information such as an operation target (for example, the robot arm AR, the end effector EF, the endoscope ES), an operation type (for example, movement, rotation), an operation position, and an operation speed.

The transmission/reception unit 110 may acquire surgical instrument information regarding the surgical instrument 30. The surgical instrument information may include the insertion distance of the surgical instrument 30 to the subject PS. The insertion distance corresponds, for example, to the distance between the platform 40 into which the surgical instrument 30 is inserted and the distal end position of the surgical instrument 30. For example, the surgical instrument 30 may be provided with a scale indicating the insertion distance of the surgical instrument 30. The transmission/reception unit 110 may electronically read the scale to obtain the insertion distance of the surgical instrument 30. In this case, for example, a linear encoder (reading device) may be attached to the platform 40, and the surgical instrument 30 may be provided with an encoding marker. The transmission/reception unit 110 may acquire the insertion distance of the surgical instrument 30 as the operator reads the scale and inputs the insertion distance via the UI 120.

The information from the surgical robot 300 may include information regarding the imaging by the endoscope ES (endoscopic information). The endoscopic information may include an image captured by the endoscope ES (actual endoscopic image) and additional information regarding the actual endoscopic image (imaging position, imaging orientation, imaging viewing angle, imaging range, imaging time, and the like).

The transmission/reception unit 110 may acquire information from the overview camera 400. The information from the overview camera 400 may include a captured image captured by the overview camera 400 and the additional information regarding the captured image. The additional information may include imaging-related information such as imaging position, imaging orientation, imaging viewing angle, imaging range, imaging time, and the like.

The UI 120 may include, for example, a touch panel, a pointing device, a keyboard, or a microphone. The UI 120 receives any input operation from the operator of the robotically-assisted surgical device 100. Operators may include doctors, nurses, radiologists, students, and the like.

The UI 120 receives various operations. For example, an operation, such as designation of a region of interest (ROI) or setting of a brightness condition (for example, window width (WW) or window level (WL)), in the volume data or in an image (for example, a three-dimensional image or a two-dimensional image which will be described later) based on the volume data, is received. The ROI may include regions of various tissues (for example, blood vessels, organs, viscera, bones, and brain). The tissue may include diseased tissue, normal tissue, tumor tissue, and the like.

The display 130 may include an LCD, for example, and displays various types of information. The various types of information may include a three-dimensional image and a two-dimensional image obtained from the volume data. The three-dimensional images may include a volume rendering image, a surface rendering image, a virtual endoscopic image, a virtual ultrasound image, a CPR image, and the like. The volume rendering images may include a RaySum image, an MIP image, a MinIP image, an average value image, a raycast image, and the like. The two-dimensional images may include an axial image, a sagittal image, a coronal image, an MPR image, and the like.

The memory 150 includes various primary storage devices such as ROM and RAM. The memory 150 may include a secondary storage device such as HDD or SSD. The memory 150 may include a tertiary storage device such as a USB memory, an SD card, or an optical disk. The memory 150 stores various types of information and programs. The various types of information may include volume data acquired by the transmission/reception unit 110, images generated by the processor 140, setting information set by the processor 140, and various programs. The memory 150 is an example of a non-transitory recording medium in which a program is recorded.

The processor 140 may include a CPU, a DSP, or a GPU. The processor 140 functions as a processing unit 160 that performs various types of processing and controls by executing the program stored in the memory 150.

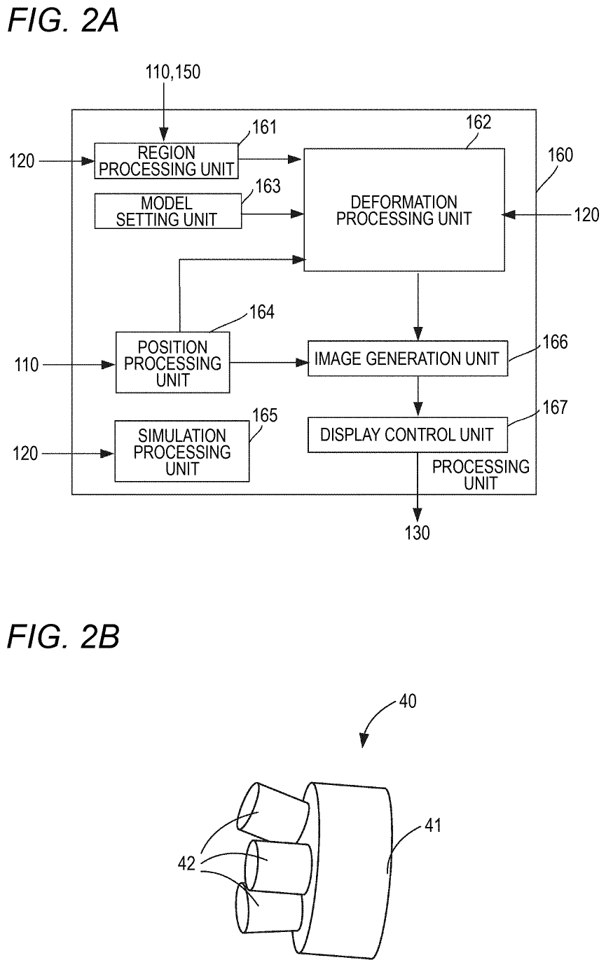

FIG. 2A is a block diagram illustrating a functional configuration example of the processing unit 160. The processing unit 160 includes a region processing unit 161, a deformation processing unit 162, a model setting unit 163, a position processing unit 164, a simulation processing unit 165, an image generation unit 166, and a display control unit 167. Each unit included in the processing unit 160 may be realized as different functions by one piece of hardware, or may be realized as different functions by a plurality of pieces of hardware. Each unit included in the processing unit 160 may be realized by a dedicated hardware component.

The region processing unit 161 acquires the volume data of the subject via the transmission/reception unit 110, for example. The region processing unit 161 extracts any region included in the volume data. The region processing unit 161 may automatically designate the ROI and extract the ROI based on a pixel value of the volume data, for example. The region processing unit 161 may manually designate the ROI and extract the ROI via the UI 120, for example. The ROI may include regions such as organs, bones, blood vessels, affected parts (for example, diseased tissue or tumor tissue). Organs may include rectum, colon, prostate, and the like.

The ROI may be segmented (divided) and extracted including not only a single tissue but also tissues around the tissue. For example, in a case where the organ which is the ROI is the rectum, not only the rectum itself, but also blood vessels that are connected to the rectum or run in or in the neighborhood of the rectum, bones (for example, spine, pelvis) or muscles neighbor of the rectum, may also be included. The above-described rectum itself, the blood vessels in or in the neighborhood of the rectum, and the bones or muscles neighbor of the rectum may be segmented and obtained as separate tissues.

The model setting unit 163 sets a model of the tissue. The model may be set based on the ROI and the volume data. The model visualizes the tissue visualized by the volume data in a simpler manner than the volume data. Therefore, the data amount of the model is smaller than the data amount of the volume data corresponding to the model. The model is a target of deformation processing and deforming operation imitating various treatments in surgery, for example. The model may be, for example, a simple bone deformation model. In this case, the model deforms the bone by assuming a frame in a simple finite element and moving the vertices of the finite element. The deformation of the tissue can be visualized by following the deformation of the bone. The model may include an organ model imitating an organ (for example, rectum). The model may have a shape similar to a simple polygon (for example, a triangle), or may have other shapes. The model may be, for example, a contour line of the volume data indicating an organ. The model may be a three-dimensional model or a two-dimensional model. The bone may be visualized by the deformation of the volume data instead of the deformation of the model. This is because, since the bone has a low degree of freedom of deformation, visualization is possible by affine deformation of the volume data.

The model setting unit 163 may acquire the model by generating the model based on the volume data. A plurality of model templates may be predetermined and stored in the memory 150 or an external server. The model setting unit 163 may acquire a model by acquiring one model template among a plurality of model templates prepared in advance from the memory 150 or the external server in accordance with the volume data.

The model setting unit 163 may set the position of a target TG in the tissue (for example, an organ) of the subject PS included in the volume data. Otherwise, the model setting unit 163 may set the position of the target TG in the model imitating the tissue. The target TG is set in any tissue. The model setting unit 163 may designate the position of the target TG via the UI 120. The position of the target TG (for example, affected part) treated in the past for the subject PS may be stored in the memory 150. The model setting unit 163 may acquire and set the position of the target TG from the memory 150. The model setting unit 163 may set the position of the target TG depending on the surgical procedure. The surgical procedure indicates a method of surgery for the subject PS. The target position may be the position of the region of the target TG having a certain size.

The deformation processing unit 162 performs processing related to the deformation in the subject PS which is a surgery target. For example, the tissue of an organ or the like in the subject PS can be subjected to various deforming operations by the operator by imitating various treatments performed by the operator in surgery. The deforming operation may include an operation of lifting an organ, an operation of flipping an organ, an operation of cutting an organ, and the like. In response to this, the deformation processing unit 162 deforms the model corresponding to the tissue of an organ or the like in the subject PS. For example, an organ can be pulled, pushed, or cut by the end effector EF, but may be simulated by deforming the model in this manner. When the model deforms, the targets in the model may also deform.

The deformation by the deforming operation may be performed with respect to the model and may be a large deformation simulation using the finite element method. For example, movement of an organ due to the body position change may be simulated. In this case, the elastic force applied to the contact point of the organ or the disease, the rigidity of the organ or the disease, and other physical characteristics may be taken into consideration. In the deformation processing with respect to the model, the computation amount is reduced as compared with the deformation processing with respect to the volume data. This is because the number of elements in the deformation simulation is reduced. The deformation processing with respect to the model may not be performed, and the deformation processing may be directly performed with respect to the volume data.

The deformation processing unit 162 may perform the gas injection simulation in which gas is virtually injected into the subject PS through the anus, for example, as processing related to the deformation. The specific method of the gas injection simulation may be a known method, and for example, a pneumoperitoneum simulation method described in Takayuki Kitasaka, Kensaku Mori, Yuichiro Hayashi, Yasuhito Suenaga, Makoto Hashizume, and Jun-ichiro Toriwaki, “Virtual Pneumoperitoneum for Generating Virtual Laparoscopic Views Based on Volumetric Deformation”, MICCAI (Medical Image Computing and Computer-Assisted Intervention), 2004, P559-P567 may be applied to the gas injection simulation in which gas is injected through the anus.

In other words, the deformation processing unit 162 may perform the gas injection simulation based on the model or the volume data of the non-gas injection state, and generate the model or the volume data of the virtual gas injection state. The volume data captured after actually injecting gas or a model based on the volume data may be used. The gas injection simulation in which the gas injection amount is changed may be performed based on the volume data captured after actually the gas is injected or the model based on the volume data. By the gas injection simulation, the operator can observe the state where gas is virtually injected, by assuming that the subject PS is in a state where gas is injected without actually injecting gas into the subject PS. Of the gas injection states, a gas injection state estimated by the gas injection simulation may be referred to as a virtual gas injection state, and a state where gas is actually injected may be referred to as an actual gas injection state.

The gas injection simulation may be a large deformation simulation using the finite element method. In this case, the deformation processing unit 162 may segment the body surface containing the subcutaneous fat of the subject PS and an internal organ near the anus of the subject PS, via the region processing unit 161. The deformation processing unit 162 may model the body surface as a two-layer finite element of skin and body fat, and model the internal organ near the anus as a finite element, via the model setting unit 163. The deformation processing unit 162 may segment, for example, the rectum and bones in any manner, and add the segmented result to the model. A gas region may be provided between the body surface and the internal organ near the anus, and the gas injection region may be expanded (swollen) in response to the virtual gas injection. The gas injection simulation may not be performed.

The position processing unit 164 derives the positional relationship between the surgical robot 300 and the platform 40. The positional relationship between the surgical robot 300 and the platform 40 may be, for example, a relative position of the platform 40 with respect to the surgical robot 300. The position processing unit 164 may derive the positional relationship by acquiring the position of the platform 40 with respect to the position of the surgical robot 300. The position processing unit 164 may derive the positional relationship by acquiring the position of the surgical robot 300 with respect to the platform 40. For example, the position processing unit 164 may acquire both the position of the surgical robot 300 and the position of the platform 40 and calculate the positional relationship. The position processing unit 164 may calculate the position of the platform 40 based on the positional relationship between the surgical robot 300 and the platform 40 and the position of the surgical robot 300.

The position processing unit 164 may derive the positional relationship between the platform 40 and the surgical robot 300 based on the insertion distance of the surgical instrument 30 into the subject PS. For example, by subtracting the insertion distance from the length of the surgical instrument 30 included in the kinematics of the surgical robot 300, the distance between the platform 40 and the surgical robot 300 may be calculated, and the positional relationship may be derived. Furthermore, the position processing unit 164 may determine the orientation of the platform 40 with respect to the robot arm AR based on the angle of the robot arm AR. In this case, the positional relationship between the surgical robot 300 and the platform 40 can be derived in consideration of the position and orientation.

The position processing unit 164 calculates a rotation center rc when operating the surgical instrument 30 during surgery based on the positional relationship between the surgical robot 300 and the platform 40. The rotation center rc is a position through which the surgical instrument 30 passes regardless of a change in the position or orientation of the surgical instrument 30. The position of the rotation center rc may be, for example, the actual position of the platform 40 or the ideal arrangement position of the platform 40. The ideal arrangement position of the platform 40 may be the estimated position of the platform 40 based on the angle of the robot arm AR and the kinematics of the surgical instrument 30. As the operator adjusts the movement of the surgical instrument 30 to pass through the rotation center rc via the robot operation terminal 310, it is possible to suppress the unnecessary pressing force with respect to the platform 40 and reduce the burden on the subject PS during surgery. As the operator adjusts the platform 40 such that the movement of the surgical instrument 30 passes through the rotation center rc by directly touching the platform 40, it is possible to suppress the unnecessary pressing force with respect to the platform 40 and reduce the burden on the subject PS during surgery.

The position processing unit 164 may calculate the rotation center rc for each surgical instrument 30. In this case, the position on the platform 40 through which each surgical instrument 30 passes may be calculated individually. Accordingly, as the operator adjusts the movement of each of the plurality of surgical instruments 30 to respectively pass through the rotation center rc, it is possible to suppress the unnecessary pressing force with respect to the platform 40 and reduce the burden on the subject PS during surgery.

The position processing unit 164 may estimate the position through which the surgical instrument 30 attached to the robot arm AR passes, based on the information on the kinematics of the surgical instrument 30 and the angle of the robot arm AR. In this case, the angle of the end effector EF with respect to the robot arm AR may be added together with the angle of the robot arm AR. The estimated position of the end effector EF may differ from the actual position of the end effector EF. The position of the rotation center rc of the end effector EF may be the estimated position of the end effector EF. Accordingly, it is possible to grasp the ideal position of the rotation center rc of the end effector EF in a case where there is no curvature or the like of the end effector EF.

The position processing unit 164 estimates the distal end position and the distal end orientation of the surgical instrument 30. For example, the position processing unit 164 may estimate the distal end position of the surgical instrument 30 at least based on the angle (the orientation of the surgical instrument 30) of the robot arm AR and the insertion distance of the surgical instrument 30. For example, in a case where the surgical instrument 30 has a shape that extends linearly, the distal end position of the surgical instrument 30 may be estimated based on the insertion distance of the surgical instrument 30 and the angle of the robot arm AR. For example, in a case where the surgical instrument 30 has a non-linear shape (for example, a bent shape or a curved shape), the distal end position of the surgical instrument 30 may be estimated based on the angle of the robot arm AR, the insertion distance of the surgical instrument 30, and the shape of the surgical instrument 30. For example, in a case where the surgical instrument 30 has a shape that extends linearly, the orientation of the entire surgical instrument 30 may be calculated as the distal end orientation of the surgical instrument 30 based on the angle of the robot arm AR. For example, in a case where the surgical instrument 30 has a non-linear shape, the distal end orientation of the surgical instrument 30 may be calculated based on the orientation of the entire surgical instrument 30 and the shape of the surgical instrument 30. In a case where the surgical instrument 30 received the pressing force, a difference may occur between the distal end position and the distal end orientation of the surgical instrument 30 estimated above and the actual distal end position and the actual distal end orientation of the surgical instrument 30.

The simulation processing unit 165 performs an installation simulation of the platform 40. The installation simulation of the platform 40 is a simulation for determining whether or not a desired surgery on the subject PS is possible by the operator operating the robot operation terminal 310. In the simulation, the operation of the robot operation terminal 310 may be imitated by using the UI 120. In the installation simulation of the platform 40, while assuming the surgery, by operating the end effector EF inserted through the platform 40 installed in the anus in the virtual space, the operator may determine whether the end effector EF can access the target TG which is the surgery target. In other words, in the installation simulation of the platform 40, while operating the end effector EF, it may be determined whether or not the end effector EF can access the target TG without any problem. In a case where the access is possible, it is possible to determine that TAMIS using the surgical robot 300 is possible. In a case where the access is not possible, it can be determined that TAMIS using the surgical robot 300 is not possible, and in this case, this determination becomes a determination base for switching the surgery to the laparotomy. The simulation processing unit 165 may determine whether or not the target TG is included in the imaging range of the endoscope ES while operating the endoscope ES in the installation simulation of the platform 40.

Specifically, the simulation processing unit 165 acquires the volume data or model of the subject PS, the kinematics of the surgical instrument 30, the position of the target TG, and the position of the platform 40. The simulation processing unit 165 may determine whether or not the end effector EF can reach the target TG via the platform 40 based on the kinematics of the surgical instrument, the position of the target, and the position of the platform 40, and may determine whether or not TAMIS using the surgical robot 300 is possible. Furthermore, the gas injection state from the anus into the subject PS, the surgical procedure performed with respect to the subject PS, and the like may be taken into consideration.

In the kinematics, together with the movable range of the robot arm itself, the movable range of the other robot arm is defined. Therefore, as the surgical robot 300 operates each robot arm AR of the surgical robot 300 based on the kinematics, it is possible to avoid interference of the plurality of robot arms AR with each other during surgery.

The surgical procedure may be designated via the UI 120. Each treatment in the robotic surgery may be determined by the surgical procedure. Depending on the treatment, the end effector EF required for the treatment may be determined. Accordingly, the end effector EF attached to the robot arm AR may be determined depending on the surgical procedure, and it may be determined which type of end effector EF is attached to which robot arm AR.

The image generation unit 166 generates various images. The image generation unit 166 generates a three-dimensional image or a two-dimensional image based on at least a part of the acquired volume data (for example, a region extracted in the volume data). The image generation unit 166 may generate a three-dimensional image or a two-dimensional image based on the volume data deformed by the deformation processing unit 162. For example, a volume rendering image or a virtual endoscopic image visualizing a state where the orientation of the endoscope ES is viewed from the position of the endoscope ES may be generated.

The display control unit 167 causes the display 130 to display various types of data, information, and images. The display control unit 167 displays an image (for example, a rendering image) generated by the image generation unit 166. The display control unit 167 superimposes and displays various pieces of information together with the image. The superimposed and displayed information may include, for example, information indicating the surgical instrument 30. The display control unit 167 may also adjust the brightness of the rendering image. The brightness adjustment may include, for example, adjustment of at least one of a window width (WW) and a window level (WL).

FIG. 2B is a view illustrating a structural example of the platform 40. The platform 40 has a base 41 and a plurality of projections 420 connected to the base 41. The base 41 has, for example, a substantially hemispherical shape or a substantially cylindrical shape, and may have other shapes. The plurality of projections 420 have holes through which the surgical instruments 30 are respectively inserted. The holes are provided to penetrate the projection 420 and the base 41. Each surgical instrument 30 moves in conjunction with the movement of the robot arm AR, and the insertion amount (insertion distance) and insertion orientation into the subject PS can be flexibly adjusted via the projection 420 and the base 41 of the platform 40.

As the insertion distance into the subject PS, the insertion distances of the plurality of surgical instruments 30 inserted through the platform 40 may be collectively acquired as one distance. In this case, the linear encoder may be installed on the base 41, for example. The insertion distance may be acquired for each of the plurality of surgical instruments 30. In this case, the linear encoder may be installed in each projection 420.

The platform 40 is installed on the anus, but the access platform for single incisional laparoscopic surgery (SILS) can be used as is or with some modification. The platform 40 may be a platform dedicated to the anus. As for the surgical robot, a surgical robot for laparoscopic surgery can be used as the surgical robot 300 for the anus of the embodiment.

Next, the derivation of the positional relationship between the surgical robot 300 and the platform 40 will be described. Here, it is mainly exemplified that the positional relationship between the surgical robot 300 and the platform 40 is derived by using an optical marker MK. The optical marker MK may be attached to at least one of the robot main body 320, the robot arm AR, and the surgical instrument 30. The optical marker MK may be attached to at least one of the base 41 and the projection 420 of the platform 40.

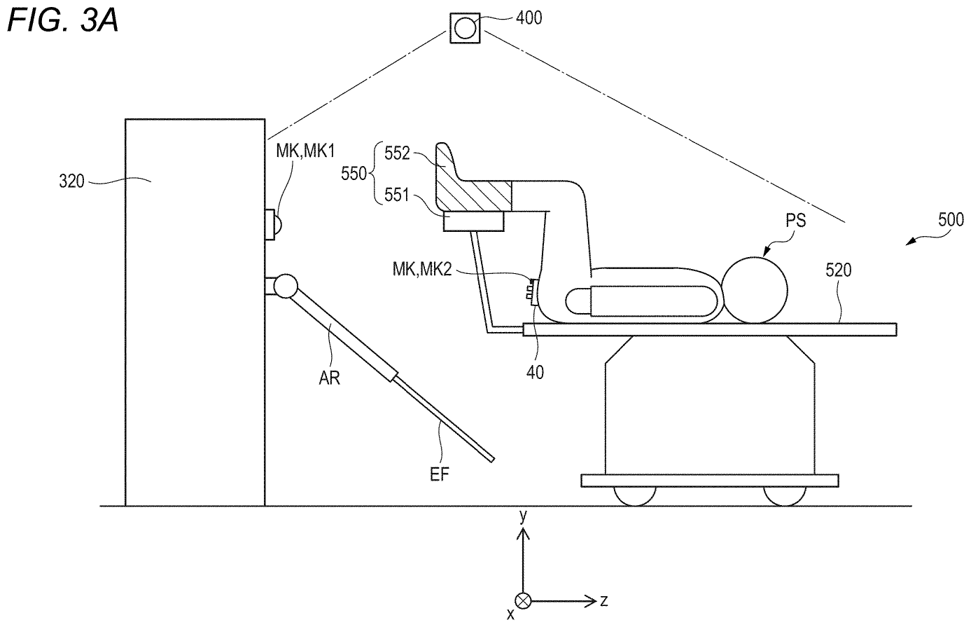

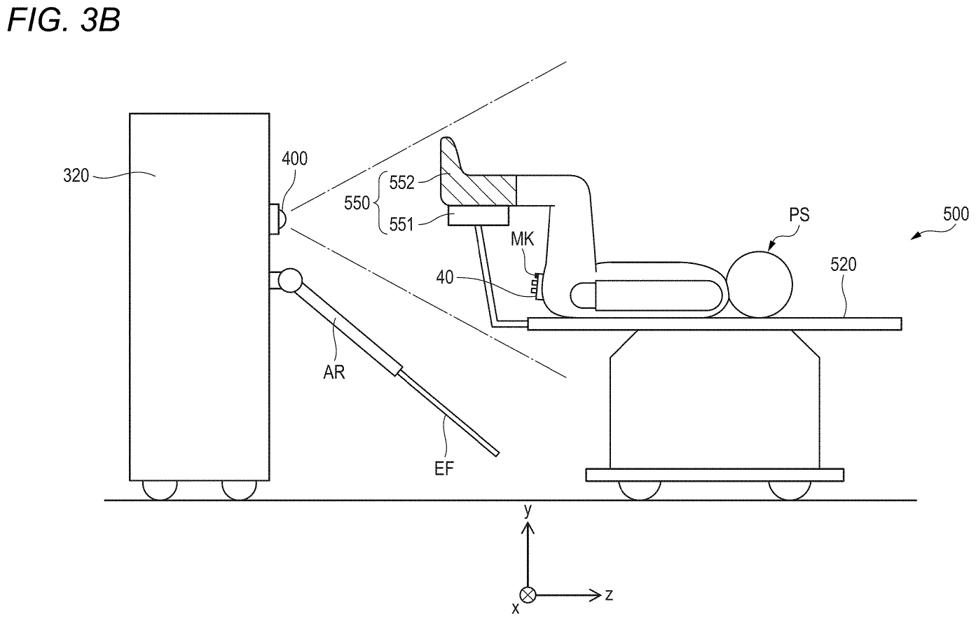

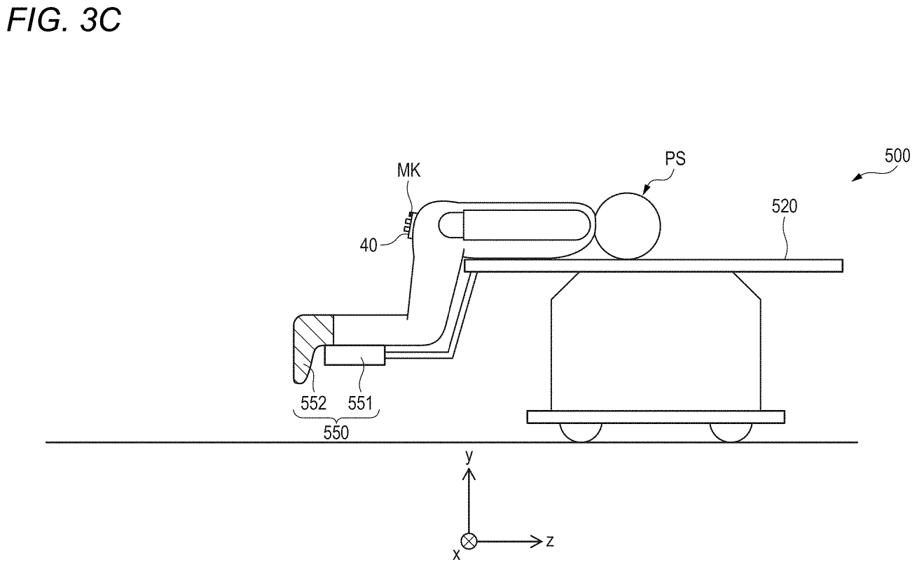

FIG. 3A is a view for describing a first derivation example of the positional relationship between the surgical robot 300 and the platform 40.

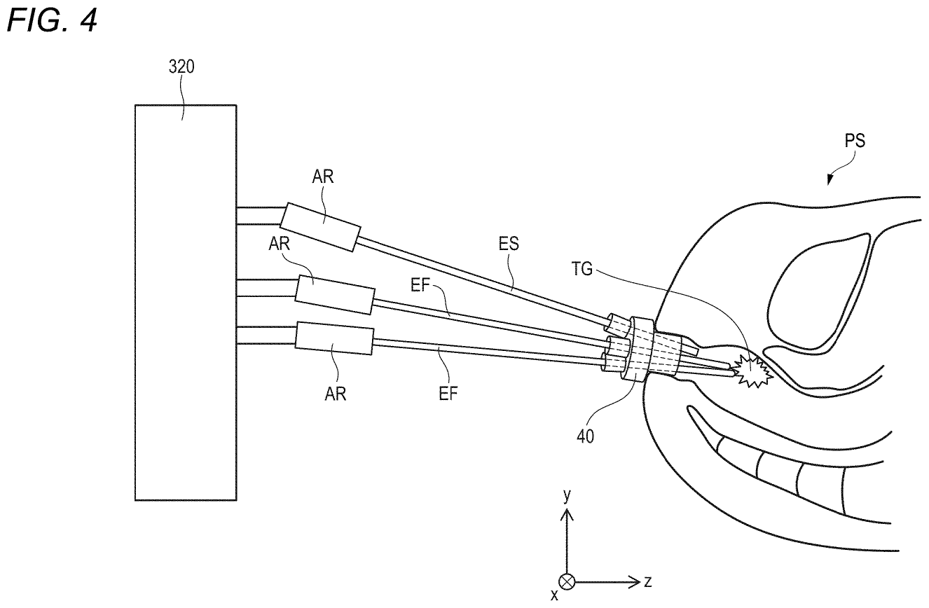

In FIG. 3A, the x-direction, the y-direction, and the z-direction of the subject coordinate system (patient coordinate system) with respect to the subject PS are illustrated. The subject coordinate system is an orthogonal coordinate system. The x-direction may be along the left-right direction with respect to the subject PS. The y-direction may be the front-rear direction (thickness direction of the subject PS) with respect to the subject PS. The z-direction may be an up-down direction (the body axial direction of the subject PS) based on the subject PS. The x-direction, the y-direction, and the z-direction may be three directions defined by digital imaging and communications in medicine (DICOM). The coordinate system is the same in FIGS. 3A to 3C, and 4.

In FIG. 3A, the optical marker MK is attached to the surgical robot 300, and the optical marker MK is attached to the platform 40. Specifically, an optical marker MK1 may be attached to a predetermined position in the surgical robot 300. The predetermined position may be, for example, the side surface of the robot main body 320 facing the place where the subject PS is placed, or the surface of the root of the robot arm AR. An optical marker MK2 may be attached to a predetermined position on the platform 40. The predetermined position may be, for example, the position of any surface on the base 41 or the projection 42.

In FIG. 3A, the overview camera 400 is arranged at a predetermined position in the surgical environment where the robotic surgery is performed. The predetermined position may be a wall surface or ceiling of the operating room, a position suspended from the ceiling, a side surface of the surgical robot 300, a side surface of various carts used in the robotic surgery, or the like. In the overview camera 400, the position, orientation, viewing angle, and the like at which the overview camera 400 is installed are determined so as to include the optical markers MK attached to the surgical robot 300 and the platform 40 in the imaging range. The optical marker MK emits light when being irradiated with infrared light from the overview camera 400 or the like. As a result, the optical marker MK is reflected in the captured image of the overview camera 400.

The position processing unit 164 of the robotically-assisted surgical device 100 acquires the captured image of the overview camera 400 and the additional information of the captured image via the transmission/reception unit 110. The position processing unit 164 recognizes the position (image position) of the surgical robot 300 in the captured image and the position (image position) of the platform 40 in the captured image, based on the positions of the optical markers MK1 and MK2 reflected in the captured image. The position processing unit 164 can recognize the position of the surgical robot 300 and the position of the platform 40 in the actual space based on the image position of the surgical robot 300 and the image position of the platform 40 in the captured image. The recognition of the position corresponds to the measurement (actual measurement) between the position of the surgical robot 300 and the position of the platform 40. Accordingly, the position processing unit 164 can derive the positional relationship between the surgical robot 300 and the platform 40.

In this manner, the position of the surgical robot 300 with the optical marker MK2 is recognized as the position of the surgical robot 300, and the position of the platform 40 with the optical marker MK1 is recognized as the position of the platform 40.

FIG. 3B is a view for describing a second derivation example of the positional relationship between the surgical robot 300 and the platform 40. In FIG. 3B, the description of the same items as in FIG. 3A will be omitted or simplified.

In FIG. 3B, the optical marker MK is not attached to the surgical robot 300, and the optical marker MK is attached to the platform 40. The position of the optical marker MK attached to the platform 40 may be the same as that in a case of FIG. 3A.

In FIG. 3B, the overview camera 400 is arranged at a predetermined position on the surgical robot 300. The predetermined position may be, for example, the side surface of the robot main body 320 facing the place where the subject PS is arranged. In the overview camera 400, the position, orientation, viewing angle, and the like at which the overview camera 400 is installed are determined so as to include the optical marker MK attached to the platform 40 in the imaging range.

The position processing unit 164 of the robotically-assisted surgical device 100 acquires the captured image of the overview camera 400 and the additional information of the captured image via the transmission/reception unit 110. Since the overview camera 400 is installed in the surgical robot 300, the image range of the captured image is based on the position of the surgical robot 300. The position processing unit 164 determines the position (image position) of the platform 40 in the captured image based on the position of the optical marker MK reflected in the captured image. Accordingly, the position processing unit 164 can estimate the position of the platform 40 with respect to the surgical robot 300 in the actual space based on the image position of the platform 40 in the captured image. Therefore, the position processing unit 164 can derive the positional relationship between the surgical robot 300 and the platform 40.

In FIGS. 3A and 3B, the subject PS is fixed to a surgical bed 500 in a lithotomy position. In the lithotomy position, the subject is placed facing upward on the surgical bed 500, and on the surgical bed 500, a leg holding unit 550 to which the leg part of the subject PS is fixed is arranged at a higher position than a table 520 on which the body part of the subject PS is placed. The leg of the subject PS is fixed to the leg holding unit 550 in a raised state. The leg holding unit 550 has a leg placing unit 551 and a leg fixing unit 552.

In the embodiment, the body position of the subject PS is not limited to the lithotomy position. For example, the body position may be the jackknife position (refer to FIG. 3C) or any other position. In the jackknife position, the subject is placed in a prone position on the surgical bed 500, and on the surgical bed 500, the leg holding unit 550 is arranged at a lower position than the table 520. The leg of the subject PS is fixed to the leg holding unit 550 in a lowered state. In FIG. 3C, the derivation example of the positional relationship illustrated in FIG. 3A may be applied, or the derivation example of the positional relationship illustrated in FIG. 3B may be applied.

FIG. 4 is a view illustrating an example of a state of the inside of the platform 40, the surgical instrument 30, and the subject PS. The end effector EF attached to the robot arm AR of the robot main body 320 is inserted into the subject PS through the platform 40. In FIG. 4, the platform 40 is installed on the anus, and reaches the target TG where the disease exists at a part of the rectum connected to the anus and the treatment is performed. The state near the target TG is imaged by the endoscope ES attached to the robot arm AR. Similar to the end effector EF, the endoscope ES is also inserted into the subject PS through the platform 40. The end effector EF for performing various treatments with respect to the target TG can be reflected on the actual endoscopic image.

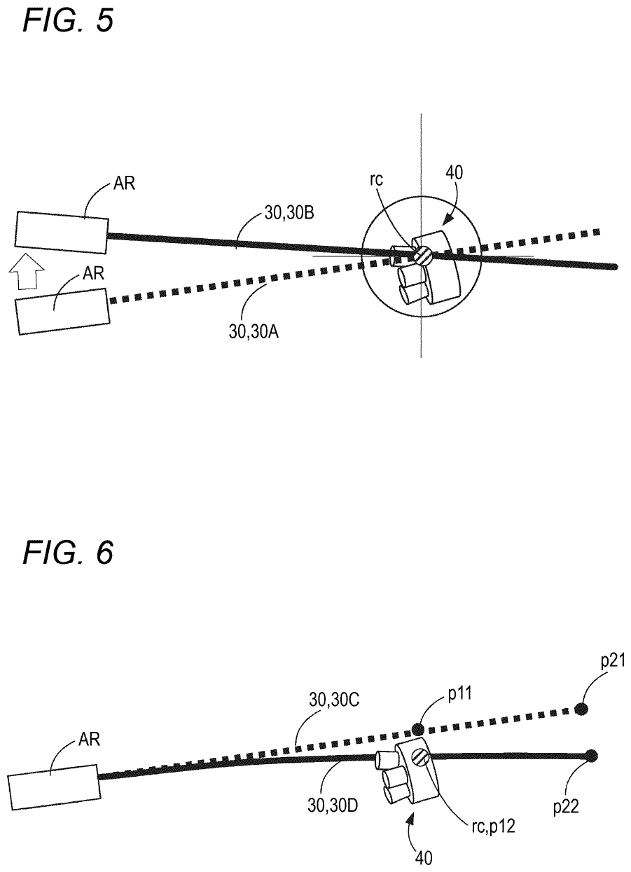

FIG. 5 is a view illustrating a rotation example of the surgical instrument 30 using the platform 40 as a rotation center.

The position processing unit 164 of the robotically-assisted surgical device 100 derives the positional relationship between the surgical robot 300 and the platform 40. The position processing unit 164 may transmit information on the positional relationship between the surgical robot 300 and the platform 40 to the surgical robot 300 via the transmission/reception unit 110. Accordingly, the surgical robot 300 can grasp the positional relationship between the surgical robot 300 and the platform 40, and can be used for the robotic surgery.

The position processing unit 164 may transmit information on the position of the rotation center rc of the surgical instrument 30 to the surgical robot 300 via the transmission/reception unit 110. The surgical robot 300 may acquire information on the position of the rotation center rc from the robotically-assisted surgical device 100, and may limit the movable range of the robot arm AR or the movable range of the surgical instrument 30 with respect to the robot arm AR so as to pass through the position (for example, the position of the platform 40) of the rotation center rc. Accordingly, in a case where the surgical instrument 30 is moved via the robot arm AR following the operation received by the robot operation terminal 310 from the operator, the surgical robot 300 can control the position of the platform 40 to be the rotation center rc of the surgical instrument 30.

In FIG. 5, the position of a surgical instrument 30A is moved to the position of a surgical instrument 30B in response to the operation of the robot operation terminal 310. Even in this case, both the surgical instruments 30A and 30B pass through the position of the rotation center rc. In other words, the surgical instruments 30A and 30B can rotate around the rotation center rc.

The display control unit 167 may display information on the positional relationship between the surgical robot 300 and the platform 40 and information on the position of the rotation center rc of the surgical instrument 30 via the display 130. In other words, the robotically-assisted surgical device 100 does not limit the movement of the robot arm AR of the surgical robot 300, and may display the above-described positional relationship or the information on the position of the rotation center rc on the display 130 or the image display terminal 330 such that the operator can recognize the information. The information on the position of the rotation center rc may be displayed as information indicating a position corresponding to the position of the rotation center rc in the rendering image, for example. The operator confirms the positional relationship or the information on the position of the rotation center rc on the display 130 or the image display terminal 330, and voluntarily limits the operation of the surgical instrument 30 via the robot operation terminal 310, and the surgical instrument 30 may pass through the rotation center rc. Accordingly, the surgical robot 300 can suppress the action of an extra pressing force on the platform 40, and the movement of the platform 40 in an unintended direction.

A plurality of positions of the platform 40 exist for each surgical instrument 30. For example, the optical markers MK are attached to each projection 42 of the platform 40 and imaged by the overview camera 400. In this case, the position processing unit 164 may allow the surgical robot 300 to recognize the position of each projection 42 on the platform 40 based on the captured image captured by the overview camera 400. The position processing unit 164 may use the position of each projection 42 as the rotation center rc of each surgical instrument 30. As the surgical robot 300 derives the position of each projection 42 as the rotation center rc of the EF of each surgical instrument 30, it is possible to adjust the position and orientation of the surgical instrument 30 with respect to the subject PS using the position of each projection 42 as the rotation center rc.

FIG. 6 is a view illustrating an example of a state where the surgical instrument 30 inserted through the platform 40 is curved.

Even in a case where the main part is linear, the surgical instrument 30 can be curved in a case where the pressing force acting on the surgical instrument 30 is excessive. Even in a case where the main part is non-linear, the surgical instrument 30 can be deformed from the original shape and curved in a case where the pressing force acting on the surgical instrument 30 is excessive. In this case, since a load is applied to the subject PS into which the surgical instrument 30 is inserted, it is desirable that the pressing force acting on the surgical instrument 30 becomes small.

The position processing unit 164 of the robotically-assisted surgical device 100 estimates the position through which the surgical instrument 30 passes near the anus of the subject PS based on the angle of the robot arm AR acquired via the transmission/reception unit 110. It is desirable that the platform 40 is positioned at the estimated position of the surgical instrument 30. In other words, it is desirable that the estimated position of the surgical instrument 30 near the anus and the actual position of the surgical instrument 30 inserted through the platform 40 match each other.

When the surgical instrument 30 is curved, the difference between an estimated position p21 of the distal end of the surgical instrument 30 and an actual position p22 of the distal end on a far side from the vicinity of the anus of the subject PS becomes larger than the difference between an estimated position p11 and an actual position p12 of the surgical instrument 30 near the anus of the subject PS. The actual position p12 of the surgical instrument 30 near the anus corresponds to the actually measured position of the platform 40. For example, in a case where the deviation is 1 to 2 mm near the anus, it is assumed that the deviation is 2 to 4 mm near the distal end of the surgical instrument 30. In FIG. 6, the non-curved surgical instrument 30 is illustrated as the surgical instrument 30C, and the curved surgical instrument 30 is illustrated as the surgical instrument 30D.

In a case where the surgical instrument 30 does not pass through the position of the platform 40, the position processing unit 164 may correct the estimated position p21 of the distal end of the surgical instrument 30 assuming that the robot arm AR or the surgical instrument 30 is curved. In this case, the position processing unit 164 may calculate the degree of curvature of the surgical instrument 30 based on the difference between the estimated position p11 of the surgical instrument 30 and the actually measured position p12 of the platform 40 near the anus. The position processing unit 164 may calculate the position p22 of the distal end of the surgical instrument 30 based on the calculated degree of curvature and the estimated position p21 of the distal end of the surgical instrument 30. The position processing unit 164 may correct the estimated position p21 of the distal end of the surgical instrument 30 based on the angle of the robot arm AR to the calculated position p22 of the distal end of the surgical instrument 30 based on the degree of curvature. The position processing unit 164 may not correct the estimated position p21 of the distal end of the surgical instrument 30 to move to reach the calculated position p22. For example, the position processing unit 164 may correct the estimated position p21 of the distal end to the calculated position p22 by any correction amount, and may correct the position to be a position between the estimated position p21 of the distal end and the calculated position p22.

Accordingly, the estimated positions p11 and p13 of the surgical instrument 30 based on the angle of the robot arm AR can be corrected according to the actual state of the surgical instrument 30 in the actual space. The result of the correction of the distal end position of the surgical instrument 30 is notified to the surgical robot 300 and recognized, and accordingly, the accuracy of the robotic surgery by the surgical robot 300 is improved. The result of the correction is superimposed and illustrated in the rendering image of the subject PS, and accordingly, the accuracy of the robotic surgery in response to the operation of the operator is improved.

The correction of the distal end position of the surgical instrument 30 may be the opposite of the above. In other words, the position processing unit 164 may correct the calculated position p22 of the distal end of the surgical instrument 30 to the estimated position p21 of the distal end of the surgical instrument 30. Accordingly, the actual position p12 and the calculated position p22 of the distal end of the surgical instrument 30 can be corrected to the estimated position p11 and the estimated position p21 of the distal end of the surgical instrument 30 that can be estimated by the surgical robot 300 itself. Accordingly, the robotically-assisted surgical device 100 causes the surgical robot 300 to recognize the result of the correction or present the result of the correction to the operator via a display or the like, and accordingly, the surgical instrument 30 can be easily guided such that the ideal position of the platform 40 at which the curvature does not occur and the pressing force is reduced is the rotation center rc.

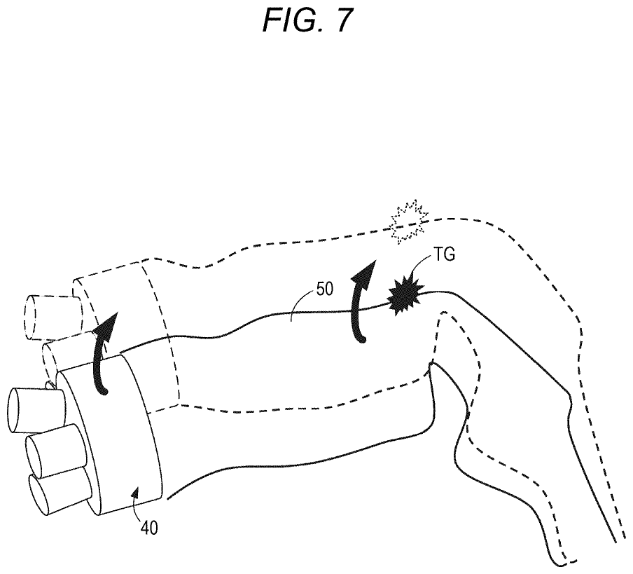

FIG. 7 is a view illustrating a movement example of an organ 50 which is in conjunction with the movement of the platform 40.

The organ 50 which is in contact with the platform 40 moves in conjunction with the movement of the platform 40. The organ 50 which is in contact with the platform 40 may include, for example, the muscles neighbor of the anus or the rectum. The position processing unit 164 derives the positional relationship between the subject PS and the platform 40. Since the position processing unit 164 makes the position of the anus of the subject PS and the position of the platform 40 installed on the anus match each other, the positional relationship between the subject PS and the platform 40 can be grasped. Since the volume data has the positional information corresponding to each voxel of the volume data, the position processing unit 164 can recognize the position of the anus of the subject PS in the volume data.

The deformation processing unit 162 calculates the deformation of the organ 50 which is in conjunction with the platform 40 of the subject PS based on the positional relationship between the subject PS and the platform 40 and the movement of the platform 40. The deformation here may include movement and rotation. Since the target TG is included in the organ 50, the deformation processing unit 162 may calculate the deformation of the target TG based on the movement of the platform 40.

By moving the platform 40 by applying a force to the platform 40, the operator or the surgical robot 300 may move the organ 50 which is in contact with the platform 40 or the target TG in the organ 50. For example, by moving the platform 40, the position of the target TG may be moved to a position where the target TG can be easily treated. The pelvis of the subject PS may be treated as a part of which the position can be changed with respect to the subject PS, or may be treated as a part of which the position is fixed with respect to the subject PS, in the volume data or the model.

The deformation processing unit 162 may acquire information on the movement of the pelvis in the subject PS. For example, the movement of the pelvis may be calculated based on the positional relationship between the subject PS and the platform 40, the movement of the platform 40, and the position of the pelvis in the subject PS. By acquiring information on the movement of the pelvis, for example, muscle peeling near the anus becomes easier. The movement of the pelvis becomes larger in the body position change by raising and lowering the leg than that in the body position change on the table 520.

Deformation and movement of the organ 50, the target TG, the pelvis, and the like may be derived by large deformation simulation using the finite element method, by using the model or the volume data. For example, the deformation processing unit 162 calculates the pressing force acting on the platform 40 in response to the movement of the platform 40, and based on the pressing force acting on each point of the organ 50 moving in conjunction with the platform 40, the organ 50 may be deformed.

The deformation processing unit 162 may acquire the position of the platform 40 before and after the subject PS changes the body position, and may calculate the movement of the platform 40 at the time of changing the body position based on the positions of the platform 40. The deformation processing unit 162 may estimate the movement of the organ or pelvis of the subject PS based on the movement of the platform 40. Accordingly, even when the body position is changed in a state (docked state) where the surgical instrument 30 is inserted into the body of the subject PS during surgery, the robotically-assisted surgical device 100 can estimate the movement of the organ 50 or the subject PS that cannot be visually recognized from the outside, and can continue the robotic surgery.

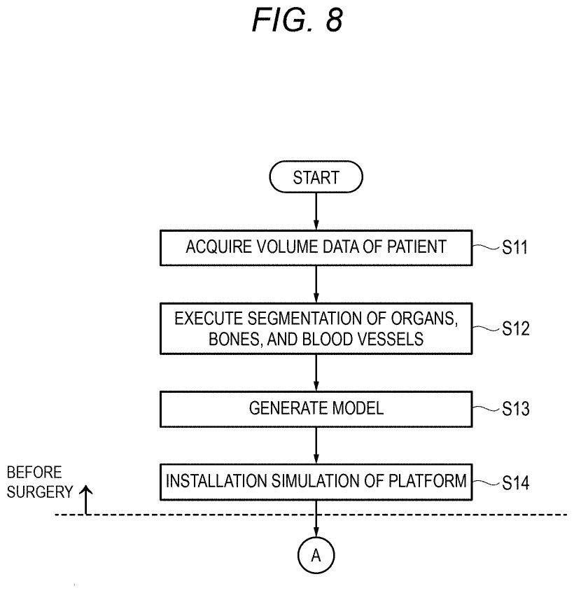

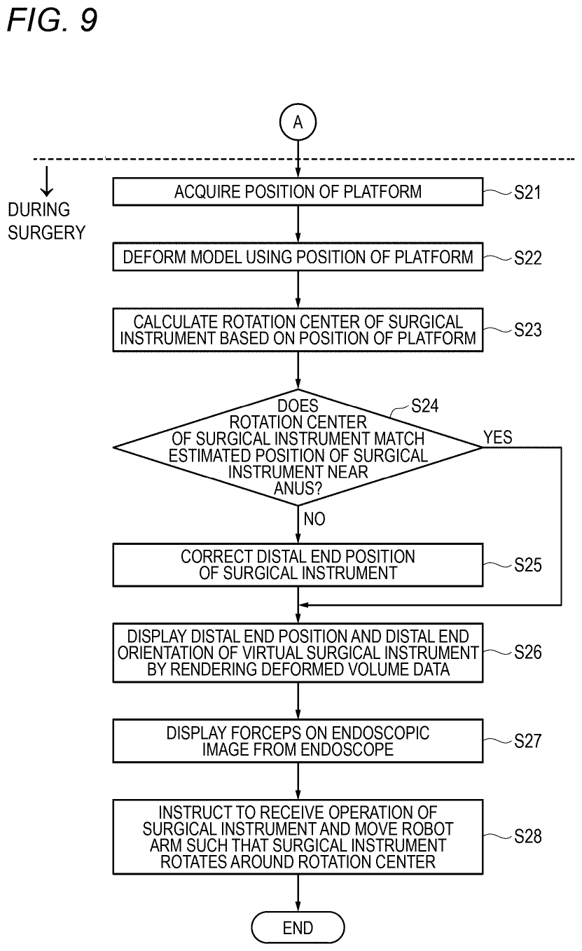

FIGS. 8 and 9 are flowcharts illustrating an operation example of the robotically-assisted surgical device 100. S11 to S14 in FIG. 8 are executed, for example, before surgery, and S21 to S28 in FIG. 9 are executed, for example, during surgery. Each processing here is executed by each part of the processing unit 160.

First, before surgery, the volume data of the subject PS (for example, a patient) is acquired (S11). Segmentation to extract regions of organs, bones, and blood vessels is executed (S12). A model (for example, a rectal organ model) is generated based on the volume data (S13). The installation simulation of the platform 40 to the anus of the subject PS is performed (S14).

When the robotic surgery is started, the surgical bed 500 on which the surgical robot 300 and the subject PS are placed is arranged at a predetermined position. During surgery, the surgical instrument 30 is inserted into the subject PS via the platform 40 installed on the anus.

Subsequently, the overview camera 400 acquires a captured image which was captured including the optical marker MK in the imaging range. The position (actually measured position) of the platform 40 is derived based on the captured image (S21). The model is deformed based on the position of the platform 40 (S22). Based on the position of the platform 40, the rotation center rc of each surgical instrument 30 is calculated (S23). It is determined whether or not the position of the rotation center rc of the surgical instrument 30 matches the estimated position of the surgical instrument 30 near the anus (S24). In a case where the position of the rotation center rc of the surgical instrument 30 does not match the estimated position of the surgical instrument 30, the estimated position p21 of the distal end of the surgical instrument 30 is corrected to be close to the calculated position p22 of the distal end of the surgical instrument 30 based on the actually measured position of the platform 40 (S25).

The volume data is deformed corresponding to the deformation of the model. For example, the deformation of the organ which is in conjunction with the platform 40 is reflected in the volume data. The deformed volume data is rendered to generate a virtual endoscopic image (S26). Information (for example, information on the distal end position and the distal end orientation) indicating the distal end of a virtual surgical instrument 30V is superimposed on the virtual endoscopic image to generate a first display image (S26). In the virtual endoscopic image, the reflected virtual surgical instrument 30V is a virtual end effector. The generated first display image is displayed on the display 130 or the image display terminal 330 (S26). The actual endoscopic image is acquired from the endoscope ES via the transmission/reception unit 110, and is displayed on the display 130 or the image display terminal 330 as a second display image (S27). The surgical instrument 30 is reflected on the actual endoscopic image.

Accordingly, the operator can grasp the internal situation of the subject PS by confirming at least one of the first display image and the second display image. For example, the state of deformation of the organ which is in conjunction with the platform 40 can be confirmed. The orientation and the distal end position of the virtual surgical instrument 30V that can be curved according to the platform 40 can be confirmed.

The robot operation terminal 310 receives the operation of the surgical instrument 30 by the operator. The transmission/reception unit 110 acquires the operation information from the robot operation terminal 310 (S28). The transmission/reception unit 110 sends an instruction to the surgical robot 300 to move the robot arm AR to which the surgical instrument 30 is connected so as to rotate around the rotation center rc of the surgical instrument 30 (S28).

When receiving the instruction, the surgical robot 300 moves the robot arm AR following the instruction (S28). At this time, for example, the operator may operate the robot operation terminal 310 so as to move the robot arm AR around the rotation center rc.

The processing of S21 to S28 may be repeated during surgery. At least a part of the processing of S11 to S14 may be repeated by imaging the patient with a cone beam CT or the like during surgery.

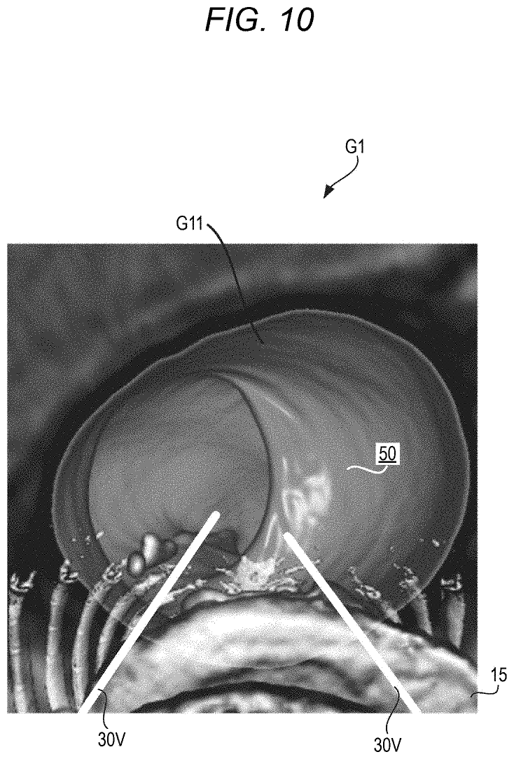

FIG. 10 is a view illustrating an example of a first display image G1. The first display image G1 includes a virtual endoscopic image G11. In the virtual endoscopic image G11, the state of the vicinity of the organ 50 (for example, the rectum) which is a tubular tissue is illustrated. The first display image G1 illustrates information indicating a bone 15 existing behind the organ 50 and information indicating the virtual surgical instrument 30V together with the virtual endoscopic image G11. In the first display image G1, the virtual surgical instrument 30V is illustrated at the image position in the virtual endoscopic image G11 corresponding to the position of the surgical instrument 30 in the actual space.

In this manner, the robotically-assisted surgical device 100 can derive the positional relationship between the surgical robot 300 and the platform 40 even when the platform 40 used in TAMIS easily moves during surgery. The robotically-assisted surgical device 100 may iteratively derive the positional relationship between the surgical robot 300 and the platform 40, and the robot arm AR can follow the movement of the platform 40. Therefore, the operator can perform the robotic surgery without worrying about the position of the platform 40 with respect to the surgical robot 300, and can improve the surgical accuracy. In particular, in TAMIS, the movement of the organs in the subject PS is large and the deformation is large as compared with the body position change in other laparoscopic surgeries. On the other hand, the robotically-assisted surgical device 100 can suppress the influence by deriving the above-described positional relationship.

The operator or the surgical robot 300 can recognize the rotation center rc of the surgical instrument 30. Therefore, it is possible to suppress sudden limitation of the operating range that may occur while the operator does not recognize. Accordingly, the operator can suppress the action of an unnecessary pressing force on the platform 40 without worrying about the rotation center rc, and can suppress the reception of the pressing force by the surgical instrument 30 via the platform 40.

Although various embodiments have been described above with reference to the drawings, it is needless to say that the present disclosure is not limited to such examples. It is clear that a person skilled in the art can come up with various changes or modifications within the scope of the claims, and it is understood that these changes or modifications naturally belong to the technical scope of the present disclosure.

The platform 40 may have a position sensor instead of the optical marker MK. The position sensor may be a three-dimensional position sensor. The robot main body 320 may have a position sensor instead of the optical marker MK. The position sensor may be a three-dimensional position sensor. These position sensors may detect the position optically or magnetically. The platform 40 and the robot main body 320 may be directly connected to detect the position of the platform 40 with respect to the robot main body 320. Specifically, the position sensor may be attached to the robot arm AR or the surgical instrument 30 of the surgical robot 300. The position sensor may include a three-dimensional position sensor that detects the position of the robot arm AR or the surgical instrument 30 in the three-dimensional space.

The transmission/reception unit 110 may acquire information from the platform 40. A position sensor or an angle sensor may be attached to the platform 40. The position sensor may include a three-dimensional position sensor that detects the position of the platform 40 in the three-dimensional space. The angle sensor may be an angle sensor that detects the orientation of the platform 40 with respect to the ground surface or the orientation with respect to the subject PS, or may be a three-axis angle sensor. The transmission/reception unit 110 may acquire the detection information detected by various sensors attached to the platform 40. The information from the platform 40 may include the shape information regarding the shape of the platform 40.

The optical markers MK may be attached to the platform 40 at any of the plurality of positions. Based on the image captured by the overview camera 400, each position with each optical marker MK may be detected at a plurality of different time points. The deformation processing unit 162 may detect the movement of the plurality of positions on the platform 40 corresponding to the positions of the plurality of optical markers MK based on each position with each optical marker MK detected at a plurality of different time points, that is, may detect the deformation of the platform 40. By detecting the deformation of the platform 40, the robotically-assisted surgical device 100 can estimate the position or the orientation at which the pressing force acts on the platform 40 and the magnitude of the pressing force, and can be expected to reduce the load on the subject PS in the robotic surgery.

The overview camera 400 may be provided on the robot main body 320. In this case, the overview camera 400 does not need to acquire the positional relationship with the robot main body 320, and thus, the optical marker MK is not necessary. The robot main body 320 may have an overview camera arm, and the overview camera arm may be provided with the overview camera 400. In this case, the robotically-assisted surgical device 100 does not need to acquire the positional relationship between the overview camera 400 and the robot main body 320, and thus, the optical marker MK is not necessary. The robot arm AR may be provided on the overview camera arm.

Although the above-described embodiments can be applied to TAMIS, the embodiment may be applied to other surgical procedures, for example, to transanal total mesenteric excision (TaTME). The embodiments may also be applied to single-hole laparoscopic surgery.

The embodiments can be used not only for the robotic surgery based on the operation of the operator, but also for autonomous robotic surgery (ARS) or semi-ARS. ARS is a fully automatic robotic surgery performed by an AI-equipped surgical robot. Semi-ARS basically automatically performs the robotic surgery by an AI-equipped surgical robot, and partially performs the robotic surgery by the operator.

Although the endoscopic surgery by the robotic surgery is exemplified, the surgery may be performed by the operator directly operating the surgical instrument 30. In this case, the robot main body 320 may be the subject PS, the robot arm AR may be the arm of the operator, and the surgical instrument 30 may be forceps and an endoscope that the operator grasps and uses for treatment.

The preoperative simulation and the intraoperative navigation may be configured by a separate robotically-assisted surgical device. For example, the preoperative simulation may be performed by a simulator, and the intraoperative navigation may be performed by a navigator.

The robotically-assisted surgical device 100 may include at least the processor 140 and the memory 150. The transmission/reception unit 110, the UI 120, and the display 130 may be externally attached to the robotically-assisted surgical device 100.

It is exemplified that the volume data as the captured CT image is transmitted from the CT scanner 200 to the robotically-assisted surgical device 100. Instead of this, the volume data may be transmitted to and stored in a server (for example, an image data server (PACS) (not illustrated)) or the like on the network such that the volume data is once stored. In this case, the transmission/reception unit 110 of the robotically-assisted surgical device 100 may acquire the volume data from a server or the like via a wired circuit or a wireless circuit when necessary, or may acquire the volume data via any storage medium (not illustrated).

It is exemplified that the volume data as the captured CT image is transmitted from the CT scanner 200 to the robotically-assisted surgical device 100 via the transmission/reception unit 110. This also includes a case where the CT scanner 200 and the robotically-assisted surgical device 100 are established by being substantially combined into one product. This also includes a case where the robotically-assisted surgical device 100 is handled as the console of the CT scanner 200. The robotically-assisted surgical device 100 may be provided in the surgical robot 300.

Although it is exemplified that the CT scanner 200 is used to capture an image and the volume data including information on the inside of the subject is generated, the image may be captured by another device to generate the volume data. Other devices include a magnetic resonance imaging (MRI) device, a positron emission tomography (PET) device, a blood vessel imaging device (angiography device), or other modality devices. The PET device may be used in combination with other modality devices.

A robotically-assisted surgical method in which the operation in the robotically-assisted surgical device 100 is defined can be visualized. A program for causing a computer to execute each step of the robotically-assisted surgical method can be visualized.

### Overview of Above-Described Embodiments

In one aspect of the above-described embodiments, the robotically-assisted surgical device 100 assists the endoscopic surgery by the surgical robot 300, and includes the processing unit 160. The processing unit 160 has a function of deriving the positional relationship between the platform 40 (an example of an access platform) installed on the subject PS which is the surgery target and capable of inserting at least two surgical instruments 30 and the surgical robot 300.

Accordingly, the robotically-assisted surgical device 100 can derive the positional relationship between the surgical robot 300 and the platform 40 even when the platform 40 easily moves during surgery, and can follow the platform 40 with respect to the surgical robot 300 or the robot arm AR. Therefore, the operator can perform the robotic surgery without worrying about the position of the platform 40 with respect to the surgical robot 300, and can improve the surgical accuracy. Accordingly, the robotically-assisted surgical device 100 can improve the operability and safety of the operation using the platform 40.

The processing unit 160 may calculate the rotation center rc of the surgical instrument 30 based on the positional relationship between the platform 40 and the surgical robot 300. The rotation center rc is a virtual rotation center rc, and may correspond to, for example, the actual position of the platform 40 or the ideal position where the platform 40 should be arranged. The surgical robot 300 can suppress the action of the unnecessary pressing force on the actual platform 40 by adjusting the movable range (position or orientation) of the robot arm AR such that the surgical instrument 30 passes through the rotation center rc. By using the ideal rotation center rc as a reference, it can be expected that the unnecessary pressing force acting at the current position of the platform 40 can be reduced. Accordingly, the robotically-assisted surgical device 100 can smooth the movement of the surgical instrument 30 and the movement of the wire passing through the inside of the surgical instrument 30, and improve the position accuracy of the surgical instrument 30 and the accuracy of the gripping force or the like of the surgical instrument 30. Accordingly, the burden on the subject PS during surgery can be reduced.

The processing unit 160 may calculate different rotation centers rc for each surgical instrument 30 based on the positional relationship between the platform 40 and the surgical robot 300. The position (for example, the position of the projection 42) where the surgical instrument 30 is inserted on the platform 40 is different. Accordingly, the robotically-assisted surgical device 100 can set a more appropriate position for each surgical instrument 30 as the rotation center rc by deriving the rotation center rc in consideration of the insertion position of the surgical instrument 30, and can further reduce the pressing force acting on the platform 40.

The processing unit 160 may derive the positional relationship between the subject PS and the platform 40 based on the 3D data of the subject PS and the positional relationship between the platform 40 and the surgical robot 300. The 3D data of the subject PS may include the volume data or the model of the subject PS. The volume data or the model may be in the non-gas injection state or the gas injection state. The 3D data may be surface data generated from the volume data.

Accordingly, the robotically-assisted surgical device 100 can recognize the position corresponding to the installation position of the platform 40 in the 3D data, and can recognize how the platform 40 moves in the 3D data.

The platform 40 may be installed on the anus of the subject PS. The processing unit 160 may calculate the deformation of the organ 50 of the subject PS based on the positional relationship between the platform 40 and the subject PS. Accordingly, the robotically-assisted surgical device 100 can recognize the state of the organ 50 in the subject PS corresponding to the positional relationship between the platform 40 and the subject PS. For example, as the subject PS changes the body position, even when the position of the platform 40 in the subject PS moves, the state of the organ 50 that moves following the movement can be recognized. Accordingly, the robotically-assisted surgical device 100 can assist the robotic surgery in consideration of the state of the organ 50.

The processing unit 160 may acquire information on the insertion distance by which the surgical instrument 30 is inserted into the platform 40. Accordingly, the robotically-assisted surgical device 100 can recognize the distance between the platform 40 and the distal end position of the surgical instrument 30 in the subject PS, and can assist the operator in predicting the state (for example, the distance between the surgical instrument 30 and the target TG) of the surgical instrument 30 in the subject PS.

The processing unit 160 may derive the positional relationship between the platform 40 and the surgical robot 300 based on the information on the insertion distance. Accordingly, the robotically-assisted surgical device 100 can derive the position of the platform 40 with respect to the surgical robot 300 without using the three-dimensional position sensor.

The processing unit 160 may acquire information on kinematics of the surgical robot 300, and may calculate a distal end position of the surgical instrument 30 based on the information on the kinematics of the surgical robot 300 and the positional relationship between the platform 40 and the surgical robot 300. The robotically-assisted surgical device 100 can estimate the position of the surgical instrument 30 based on, for example, the angle of the robot arm AR and the shape information of the surgical instrument 30, and can estimate the distal end position of the surgical instrument 30. Meanwhile, in an actual situation, there is a case where the estimated position of the surgical instrument 30 does not pass through the position of the platform 40. Even in this case, the robotically-assisted surgical device 100 can calculate the estimated position of the distal end of the surgical instrument 30 as a position where the distal end of the surgical instrument 30 is expected to actually exist, by considering the actual positional relationship between the platform 40 and the surgical robot 300. Accordingly, the robotically-assisted surgical device 100 can derive the distal end position of the surgical instrument 30 with high accuracy, and can improve the surgical accuracy of the robotic surgery.

According to another aspect of the above-described embodiment, there is provided a robotically-assisted surgical method for assisting endoscopic surgery by the surgical robot 300, the method including: a step of deriving the positional relationship between the platform 40 (an example of the access platform) installed on the subject PS which is a surgery target and capable of inserting at least two surgical instruments 30 and the surgical robot 300.

According to still another aspect of the embodiment, there is provided a program for causing a computer to execute the above-described robotically-assisted surgical method.

The present disclosure is useful for a robotically-assisted surgical device, a robotically-assisted surgical method, and a system that can improve the operability and safety in surgery using a platform installed on a subject.

1. A robotically-assisted surgical device that assists endoscopic surgery by a surgical robot, the robotically-assisted surgical device comprising:
a processor, wherein
the processor is configured to derive a positional relationship between the surgical robot and an access platform installed on a subject which is a surgery target and capable of inserting at least two surgical instruments.

2. The robotically-assisted surgical device according to claim 1, wherein
the processor is configured to calculate a rotation center of the surgical instrument based on the positional relationship between the surgical robot and the access platform.

3. The robotically-assisted surgical device according to claim 1, wherein
the processor is configured to calculate a rotation center which is different for each surgical instrument based on the positional relationship between the surgical robot and the access platform.

4. The robotically-assisted surgical device according to claim 1, wherein the processor is configured to:
acquire 3D data of the subject; and
derive a positional relationship between the subject and the access platform based on the 3D data of the subject and the positional relationship between the surgical robot and the access platform.

5. The robotically-assisted surgical device according to claim 4, wherein
the access platform is installed on an anus of the subject, and
the processor is configured to calculate deformation of an organ of the subject based on the positional relationship between the subject and the access platform.

6. The robotically-assisted surgical device according to claim 1, wherein
the processor is configured to acquire information on an insertion distance by which the surgical instrument is inserted into the access platform.

7. The robotically-assisted surgical device according to claim 6, wherein
the processor is configured to derive the positional relationship between the surgical robot and the access platform based on the information on the insertion distance.

8. The robotically-assisted surgical device according to claim 1, wherein the processor is configured to:
acquire information on kinematics of the surgical robot; and
calculate a distal end position of the surgical instrument based on the information on the kinematics of the surgical robot and the positional relationship between the surgical robot and the access platform.

9. A robotically-assisted surgical method for assisting endoscopic surgery by a surgical robot, the robotically-assisted surgical method comprising:
deriving a positional relationship between an access platform installed on a subject which is a surgery target and capable of inserting at least two surgical instruments and the surgical robot.

10. The robotically-assisted surgical method according to claim 9, comprising
calculating a rotation center of the surgical instrument based on the positional relationship between the surgical robot and the access platform.

11. The robotically-assisted surgical method according to claim 9, comprising:
acquiring 3D data of the subject; and
deriving a positional relationship between the subject and the access platform based on the 3D data of the subject and the positional relationship between the surgical robot and the access platform.

12. The robotically-assisted surgical method according to claim 11, wherein
the access platform is installed on an anus of the subject, and
the method comprises calculating deformation of an organ of the subject based on the positional relationship between the subject and the access platform.

13. A system comprising:
a surgical robot; and
a robotically-assisted surgical device that assists endoscopic surgery by the surgical robot, wherein the robotically-assisted surgical device comprises:
a processor, wherein
the processor is configured to derive a positional relationship between the surgical robot and an access platform installed on a subject which is a surgery target and capable of inserting at least two surgical instruments.

14. The system according to claim 13, wherein
the processor is configured to calculate a rotation center of the surgical instrument based on the positional relationship between the surgical robot and the access platform.

15. The system according to claim 13, wherein
the processor is configured to calculate a rotation center which is different for each surgical instrument based on the positional relationship between the surgical robot and the access platform.

16. The system according to claim 13, comprising
a computed tomography scanner,
wherein the processor is configured to:
acquire 3D data of the subject by using the computed tomography scanner; and
derive a positional relationship between the subject and the access platform based on the 3D data of the subject and the positional relationship between the surgical robot and the access platform.

17. The system according to claim 16, wherein
the access platform is installed on an anus of the subject, and
the processor is configured to calculate deformation of an organ of the subject based on the positional relationship between the subject and the access platform.

18. The system according to claim 13, wherein
the processor is configured to acquire information on an insertion distance by which the surgical instrument is inserted into the access platform.

19. The system according to claim 17, wherein
the processor is configured to derive the positional relationship between the surgical robot and the access platform based on the information on the insertion distance.

20. The system according to claim 13, wherein the processor is configured to:
acquire information on kinematics of the surgical robot; and
calculate a distal end position of the surgical instrument based on the information on the kinematics of the surgical robot and the positional relationship between the surgical robot and the access platform.
